(19) United States
(12) Patent Application Publication
(10) Pub. No.: US 2007/0232595 A1
(43) Pub. Date: Oct. 4, 2007

(54) Method for accelerating cutaneous barrier recovery

(75) Inventors: Kazuyuki Ikeyama, Yokohama-shi (JP); Shigeyoshi Fuziwara, Yokohama-shi (JP); Mitsuhiro Denda, Yokohama-shi (JP)

Correspondence Address: SNIDER & ASSOCIATES, P. O. BOX 27613, WASHINGTON, DC, 20038-7613, US

(73) Assignee: Shiseido Company, Ltd., Tokyo (JP)

(21) Appl. No.: 11/727,436

(22) Filed: Mar. 27, 2007

Related U.S. Application Data
(60) Provisional application No. 60/786,717, filed on Mar. 29, 2006.

(51) Int. Cl. A61K 31/542; A61K 31/5383; A61K 31/498

(52) U.S. Cl. 514/224.5; 514/250; 514/229.5

(57) ABSTRACT

A method for accelerating cutaneous barrier recovery and a method for preventing epidermal hyperplasia by inhibiting production of Nitric Oxide by an epidermal cell are provided.

### FIELD OF THE INVENTION

The present invention relates to a method for accelerating cutaneous barrier recovery.

### BACKGROUND ART

One of the most important roles of the skin for terrestrial mammals is to act as a water-impermeable barrier, preventing excess transcutaneous water loss. A decline in barrier function often parallels increased severity of clinical symptomatology(Elias and Feingold, 2001). When the stratum corneum barrier is damaged, a series of homeostatic processes is immediately accelerated, and acts to restore the barrier(Elias and Feingold, 2001). These processes include lipid synthesis, lipid processing, and the acceleration of exocytosis of lamellar bodies that contain intercellular lipids(Elias and Feingold, 2001). Previous studies suggested that lamellar body secretion and barrier recovery are influenced by the calcium gradient in the epidermis. We found that influx of calcium ions into epidermal keratinocytes reduced the secretion of epidermal lamellar bodies and delayed barrier recovery(Denda et al, 2003). This result suggested that the ion flux is critical for the first phase of the barrier recovery process.

Nitric oxide (NO) is a cell-signaling molecule that has both cytostatic and cytotoxic actions in skin(Bruch-Gerharz et al, 1998; Weller, 2003). Taniuchi et al. indicated that NO may be involved in the pathogenesis of erythema in the skin of patients with atopic dermatitis(Taniuchi et al, 2001). In psoriatic skin that shows barrier dysfunction and epidermal hyperplasia, NO is generated at high levels by epithelial keratinocytes in response to interferon-gamma and tumor necrosis factor-alpha(Giustizieri et al, 2002). NO is synthesized by three types of NO synthases (NOS), neuronal NOS (nNOS), endothelial NOS (eNOS) and inducible NOS (iNOS), all of which are expressed in the skin(Bruch-Gerharz et al, 1998; Cals-Grierson and Ormerod, 2004). Ormerod et al. demonstrated that nNOS was present through all levels of epidermis, and iNOS staining was significantly upregulated in psoriatic skin(Ormerod et al, 1998).

### DISCLOSURE OF THE INVENTION

It has been demonstrated that NO is involved in calcium dynamics. NO generated in endothelial cells and rat aortic smooth muscle cells promotes cyclic GMP (cGMP) formation, leading to a decrease of intercellular calcium concentration ([Ca²⁺]i) (Kaur et al, 1998; Lau et al, 2000). On the other hand, endogenous NO promotes intercellular calcium release from mitochondria in striatal neurons(Horn et al, 2002).

Thus, we hypothesized that skin barrier homeostasis might be improved by appropriate control of NO generation. In the present study, we first examined the effects of a NO synthase inhibitor, a nNOS inhibitor and an iNOS inhibitor on the skin barrier recovery after barrier disruption in hairless mouse. And we evaluated the barrier recovery rate after barrier disruption in nNOS^{-/-} mice. Moreover, we examined the effects of topical application of the NO donor S-nitroso-N-acetyl-D,L-penicillamine (SNAP) on the skin barrier homeostasis in hairless mouse. We also evaluated the release of NO after tape stripping of an organ culture, the effect of a NO-sensitive guanylyl cyclase inhibitor and an activator on the skin barrier recovery after barrier disruption in hairless mouse, and the effect of SNAP, a NO-sensitive guanylyl cyclase inhibitor and a calcium channel blocker on the intracellular calcium dynamics of cultured human keratinocytes. Moreover, we examined the effects of topical application of a nNOS inhibitor and a NO-sensitive guanylyl cyclase inhibitor on epidermal hyperplasia induced by barrier disruption under low environmental humidity in hairless mice.

As a result, in the first aspect, the present invention provides a method for accelerating cutaneous barrier recovery by inhibiting production of Nitric Oxide on epidermal cell.

In the second aspect, the present invention provides a method for preventing epidermal hyperplasia induced by barrier disruption by inhibiting production of Nitric Oxide on epidermal cell.

### BRIEF DESCRIPTIONS OF DRAWINGS

FIG. 1 shows the effects of topical application of NOS inhibitors in hairless mice. Topical application of L-NAME (NOS inhibitor) or nNOS inhibitor accelerated the barrier recovery of hairless mice after tape stripping. One micromolar solution of each reagent was applied on one flank, using two points per flank and four animals per treatment. **p<0.001, ***p<0.0001 compared with control. The error bar shows SD.

FIG. 2 shows the barrier recovery in nNOS knock-out mice after barrier disruption. After tape stripping, the barrier recovery in nNOS^{-/-} mice (n=4) was significantly faster than in wild-type mice (n=4). ***p<0.0001 compared with wild-type. The error bar shows SD.

FIG. 3 shows the effect of topical application of NO donor in hairless mice. Topical application of SNAP delayed the barrier recovery of hairless mice after tape stripping. One millimolar solution of SNAP was applied on one flank, using two points per flank and four animals per treatment. **p<0.001, ***p<0.0001 compared with control. The error bar shows SD.

FIG. 4 shows that NO was released from skin of hairless mice immediately after barrier disruption. The NO level was significantly increased by tape stripping. The increase was blocked by pre-incubation with nNOS inhibitor (100 μM), but not with iNOS inhibitor (100 μM). Number of animals: n=14 (control and tape) and n=12 (nNOS inhibitor and iNOS inhibitor). *p<0.05, **p<0.005, ***p<0.0005 compared with control. The error bar shows SD.

FIG. 5 shows the effects of topical application of inhibitors and activator of guanylyl cyclase (GC) in hairless mice. Topical application of a GC inhibitor and an NO-sensitive GC inhibitor accelerated the barrier recovery of hairless mice after tape stripping. One micromolar solution of each reagent was applied on one flank, using two points per flank and four animals per treatment. ***p<0.0001 compared with control. The error bar shows SD.

FIG. 6 shows that SNAP increased the intracellular calcium concentration in cultured keratinocytes. The increase was blocked by ODQ, while nifedipine (NIF) had no effect. The vertical axis shows the ratio of relative intensity (340 nm/380 nm) after treatment to that before treatment. The number of cells for each measurement was 50. ***p<0.0001 compared with control. The error bar shows SD.

FIG. 7 shows that SNAP increased the intracellular calcium concentration in the cultured keratinocytes in calcium-free medium. The vertical axis shows the ratio of relative intensity (340 nm/380 nm) after treatment to that before treatment. The number of cells for each measurement was 50. ***p<0.0001 compared with control. The error bar shows SD.

FIG. 8 shows the effect of topical application of nNOS inhibitor and ODQ on epidermal hyperplasia of hairless mice induced by barrier disruption under low environmental humidity. In (a), (b) and (c), representative sections are shown. Bars: 20 μm. Acetone treatment increased DNA synthesis in the epidermal basal layer (a: dark cells are BrdU-positive) and the increase was blocked by topical application of nNOS inhibitor (b) or ODQ (c). Bars: 20 μm. The levels of epidermal DNA synthesis and the epidermal thickness in the same experiment are shown in (d and e). The number of BrdU-positive cells and the epidermal thickness was increased by acetone treatment under dry conditions. The increase was blocked by the topical application of nNOS inhibitor and ODQ. ***p<0.0001 compared with control. The error bar shows SD.

### BEST MODE FOR CARRYING OUT THE INVENTION

The present invention is based on the discovery that NOS plays an important role as a signal of cutaneous barrier homeostasis and epidermal hyperplasia induced by barrier disruption.

The method for accelerating cutaneous barrier recovery comprises the step of inhibiting production of Nitric oxide by an epidermal cell, for example, by applying Nitric Oxide Synthase (NOS), neural Nitric Oxide Synthase (nNOS) inhibitor, Nitric Oxide sensitive guanylyl cyclase inhibitor or general cyclase inhibitor on skin.

The method for preventing epidermal hyperplasia induced by barrier disruption comprises the step of inhibiting production of Nitric oxide by an epidermal cell, for example, by applying Nitric Oxide Synthase (NOS), neural Nitric Oxide Synthase (nNOS) inhibitor, Nitric Oxide sensitive guanylyl cyclase inhibitor or general cyclase inhibitor on skin.

As the NOS inhibitor, a number of compounds are known in the art, and examples thereof include, but not limited to, L-N(G)-nitro-L-argine methyl ester (L-NAME), 7-Nitroindazole (7-NI), N⁶-(1-iminoethyl)-lysine, hydrochloride (L-NIL). N⁵-(1-iminoethyl)-L-ornithine, dihydrochloride (L-NIO), N^{G}-methyl-L-arginine, acetate salt (L-NMMA), and the like. Preferable NOS inhibitor is L-NAME.

As the neural NOS inhibitor, a number of compounds are known in the art, and examples thereof include, but not limited to, N^{ω}-propyl-L-arginine, S-methylthiocitrulline (SMTC), 7-nitroindazole, and the like. Preferable nNOS inhibitor is N^{ω}-propyl-L-arginine.

As the Nitric Oxide sensitive guanylyl cyclase inhibitor, a number of compounds are known in the art, and example thereof include, but not limited to, 1H-[1,2,4]oxadiazolo[4,3-a]quinoxalin-1-one (ODQ).

As the general guanylyl cyclase inhibitor, a number of compounds are known in the art, and examples thereof include, but not limited to, 4H-8-bromo-1,2,4-oxadiazolo[3,4-dibenz[b][1,4]oxazin-1-one, 3,7-bis(Dimethylamino)phenothiazin-5-ium, 6-anilino-5,8-quinolinedione, and the like. Preferable general cyclase inhibitor is 4H-8-bromo-1,2,4-oxadiazolo[3,4-dibenz[b][1,4]oxazin-1-one.

The above compounds themselves can be active ingredients for accelerating cutaneous barrier recovery or preventing epidermal hyperplasia induced by barrier disruption. The present invention not only ultimately leads to improvement of skin barrier functions, but can contribute to prophylaxis or treatment of dermatological diseases and cosmetic skin care.

The above compounds are used, as an active ingredient for a pharmaceutical or cosmetic composition for accelerating cutaneous barrier recovery and/or preventing epidermal hyperplasia induced by barrier disruption according to the invention, generally, as dry weight, in an amount of 0.00001 to 10% by weight, preferably 0.0001 to 5% by weight per weight of the total composition. At lower than 0.00001% by weight, the effects of the invention are hard to exert sufficiently, and even if it is compounded in an amount more than 10% by weight, so much enhancement of the effects is not attained and formulation becomes undesirably harder.

The pharmaceutical or cosmetic composition to be thus prescribed can be prepared by mixing or homogenizing the at least one of the above compounds into a suitable solvent, e.g., pure water, deionized water or buffered water, a lower alkanol such as methanol, ethanol or isopropyl alcohol or an aqueous solution thereof, glycerol or an aqueous solution thereof, a glycol such as propylene glycol or 1,3-butylene glycol or an aqueous solution thereof, or an oil such as hardened castor oil, vaseline or squalane, if necessary with use of a surfactant or the like. Into the composition can further appropriately be compounded, in such a range that the effects of the invention, that is, acceleration of cutaneous barrier recovery and/or prevention of epidermal hyperplasia induced by barrier disruption is/are not spoiled, other components usually used for external preparations such as cosmetics or pharmaceuticals, for example whitening agents, humectants, antioxidants, oily substances, ultraviolet absorbers, surfactants, thickeners, higher alcohols, powdery substances, colorants, aqueous substances, water, various skin nutrients, etc., according to necessity. Further, into the composition of the invention can appropriately be compounded sequestering agents such as disodium edetate, trisodium edetate, sodium citrate, sodium polyphosphate, sodium metaphosphate and gluconic acid, drugs such as caffeine, tannin, verapamil, tranexamic acid and its derivatives, grabridin, extract of fruit of Chinese quince with hot water, various crude drugs, tocopherol acetate, and glycylrrhetinic acid and its derivatives or salts, whiteners such as vitamin C, magnesium ascorbate phosphate, ascorbic acid glucoside, arbutin and kojic acid, saccharides such as glucose, fructose, mannose, sucrose and trehalose, vitamin A derivatives such as retinoic acid, retinol, retinol acetate and retinol palmitate, etc.

As to the above composition, its dosage form is not particularly limited, and can be any dosage forms such as solutions, solubilizing forms, emulsified forms, dispersed powders, water-oil two layer forms, water-oil-powder three layer forms, ointments, gels or aerosols. Its use form can also be optional, and can, for example be facial cosmetics such as skin lotion, liquid cream, cream and pack, foundation, and further makeup cosmetics, cosmetics for hair, aromatic cosmetics, bathing agents, etc., but is not limited thereto.

When the above composition is used on a living body, it can be endermically administered to local skin or the whole body skin of a subject. Its dose cannot be limited because the optimal amount varies depending on the age, sex and skin state of subjects, but, usually, it is sufficient that a composition prepared as mentioned above is administered onto the skin once or several times a day. If necessary, the dose or administration frequency can be determined referring to results obtained by evaluating a suitable specimen according to the evaluation method described later.

### EXAMPLES

### Materials and Methods

### Materials

Male hairless mice were purchased from Hoshino Laboratory Animals (Saitama, Japan). Male nNOS-deficient mice and C57BL/6J×129 hybrid control were purchased from Jackson Laboratories (Maine, USA). All animals were employed between 7-10 week-old. All procedures for measuring skin barrier function, disrupting the barrier and applying the sample were carried out under anesthesia. All experiments were approved by the Animal Research Committee of the Shiseido Research Center in accordance with the National Research Council Guide (National Research Council 1996). The NOS inhibitor L-N(G)-nitro-L-arginine methyl ester (L-NAME), iNOS inhibitor S-methylisothiourea, nNOS inhibitor (N^{ω}-propyl-L-arginine), general guanylyl cyclase inhibitor NS2028 (4H-8-bromo-1,2,4-oxadiazolo[3,4-dibenz[b][1,4]oxazin-1-one, NO-sensitive guanylyl cyclase inhibitor 1H-[1,2,4]oxadiazolo[4,3-a]quinoxalin-1-one (ODQ) and guanylyl cyclase activator SIN (SIN-1 chloride) were purchased from Tocris (Bristol, UK). The NO donor S-nitroso-N-acetyl-D,L-penicillamine (SNAP) was purchased from Sigma (Sigma, Tokyo, Japan). Total nitric oxide assay kit was purchased from IBL (Gunma, Japan). All the other reagents were purchased from Wako (Osaka, Japan).

### Cutaneous Barrier Function

Permeability barrier function was evaluated by measurement of transepidermal water loss (TEWL) with an electric water analyzer, as described previously(Denda et al, 1998). For barrier recovery experiments, the flank skin on both sides was subjected to repeated tape stripping until the TEWL reached 7-10 mg per cm² per h, as described previously(Denda et al, 1998). As for nNOS-deficient mice and wild-type, the TEWL of the ears was measured over the same sites at 1, 3 and 6 hours after tape stripping. This is because the hair shaving may induce barrier disruption. Therefore, the ears that are hairless parts were selected for TEWL measurements. Immediately after barrier disruption, 100 μl of aqueous solution containing 1 μM reagent or water alone (control) was applied to the treated area. We did not apply the same reagent to both flanks. The areas were covered with plastic membranes for 15 min and then the membranes were removed. Two points on one flank were measured and 4-8 mice were used to evaluate the effect of each treatment. We always disrupted the barrier between 7:00 AM and 8:00 AM and carried out the measurements of the barrier repair thereafter to avoid the influence of circadian rhythm on the repair rate(Denda and Tsuchiya, 2000). TEWL was measured over the same sites at 1, 3 and 6 hours after barrier disruption. The barrier recovery results are expressed as percent recovery, because of variations from day to day in the extent of barrier disruption. In each animal, the percentage recovery was calculated according to the following formula:

$$\frac{( \begin{matrix}TEWL immediately after barrier disruption - \\ TEWL at indicated time point\end{matrix} ) \times 100 \%}{( \begin{matrix}TEWL immediately after barrier disruption - \\ baseline TEWL\end{matrix} )}$$

### Epidermal hyperplasia induced by barrier disruption under low humidity

Hairless mice were kept separately in 7.2-liter cages in which the relative humidity was maintained at less than 10% with dry air as described previously(Denda et al, 1998). The temperature was the same in all cases (22-25° C.), and fresh air was circulated 100 times per hour. Animals were kept out of the direct stream of air. During the experiments, the animal's behavior was not restricted. The level of NH₃ was always below 1 ppm. Animals were first kept in a dry condition for 48 hours and then the skin on both flanks was treated with acetone-soaked cotton balls, as described previously(Denda et al, 1998). The procedure was terminated when TEWL reached 2.5-3.5 mg per cm² per h. Immediately after the barrier disruption, 100 μl of nNOS inhibitor and ODQ aqueous solution (1 μM) was applied on one side of the treated area. Water was applied on the other side. Then the animals were again kept in the dry condition for 48 hours. After the experiments, animals were euthanized with diethylether inhalation and skin samples were taken from the treated areas. One hour before euthanization, 20 μl per g body weight of a 10 mM solution of bromodeoxyuridine (BrdU) was injected intraperitoneally. Untreated control mice were also treated with BrdU at the same time. After fixation with 4% paraformaldehyde, full thickness skin samples were embedded in paraffin, sectioned (4 μm), and processed for hematoxylin and eosin staining. On each section, five areas were selected at random; the thickness of the epidermis was measured with an optical micrometer, and the mean value was calculated. For the assessment of DNA synthesis, the sections were immunostained with anti-BrdU antibodies. On each section, five areas were selected at random from one section; the number of immunostained cells per 1 mm of epidermis was counted and the epidermal thickness was measured with an optical micrometer. The mean value was calculated. Measurements were carried out in an observer-blinded fashion. NO evaluation in hairless mouse epidermis

Four male hairless mice (7-10 wk old) were killed by cervical dislocation under anesthesia and skin samples were taken immediately from both flanks. Subcutaneous fat was removed with a scalpel and the skin samples that contained epidermis and dermis, were cut into squares (1.5×1.5 cm²). Three pieces of skin from the two flanks of each animal were placed, epidermis side upwards, in separate 35-mm culture dishes kept in an ice/water bath, and one of them was tape-stripped four times. The other piece of skin was not treated. One milliliter of phosphate-buffered saline (PBS) was added to both dishes, which were incubated for 30 min at 37° C. After the incubation, 50 μL aliquots of the PBS were removed. NO released into the PBS was quantified using a total nitric oxide assay kit (Immuno-Biological Laboratories Co., Ltd, Gunma, Japan), according to the manufacturer's instruction.

### Calcium Dynamics in Keratinocyte Culture System

### Cells and Cell Culture

Normal human epithelial keratinocytes (NHEK) were purchased from Kurabo (Osaka, Japan). NHEK were cultured in serum-free keratinocyte growth medium, consisting of Humedia-KB2 (Kurabo, Osaka, Japan) supplemented with bovine pituitary extract (0.4% vol/vol), human recombinant epidermal growth (0.1 μg per mL), insulin (10 μg per mL), and hydrocortisol (0.5 μg per mL). The medium was replaced every 2-3 d. For the electrophysiological experiments, NHEK (passage 1-3 cells) were seeded onto collagen-coated glass coverslips and used within 4 d. Ca²⁺ imaging in single keratinocytes

NHEK were grown to approximately 80-100% confluency on collagen-coated cover glass chambers (Nalge Nunc, Naperville, Ill.). Changes in [Ca²⁺] in single cells were measured by the fura-2 method as described by Grynkiewicz et al. (1985) with minor modifications(Koizumi et al, 1998). In brief, the culture medium was replaced with a balanced salt solution (BSS) of the following composition (mM): NaCl 150, KCl 5, CaCl₂1.8, MgCl₂ 1.2, HEPES 25, NaH₂PO₄ 1.2 and D-glucose 10 (pH 7.4) or calcium-free BSS which had the same composition as the above BSS with 1 mM EGTA and no added CaCl₂. Cells were loaded with 5 μM fura-2 acetoxymethylester (fura-2AM, Molecular Probes, Eugene, Oreg.) at room temperature (21-23° C.) in BSS for 45 min, washed with BSS, and incubated for a further 30 min to allow de-esterification of the loaded dye. The cover slip was mounted on an inverted epifluorescence microscope (IX70, TS Olympus, Tokyo, Japan), equipped with a 75 W xenon lamp and band-pass filters of 340 and 380 nm. The image data, recorded by a high-sensivity CCD (charge-coupled-device) camera (ORCA-ER, Hamamatsu Photonics, Hamamatsu, Japan) were evaluated with a Ca²⁺ analyzing system (AQUACOSMOS/RATIO, Hamamatsu Photonics). SNAP (300 μM), ODQ (10 μM) and nifedipine (50 μM) were dissolved in the BSS and the cells were exposed to the solution by perfusion. We observed four lots of pooled cells for each treatment. Each microscopic field contained approximately 50-60 cells. Data were represented as the ratio of fluorescence intensities at 340 and 380 nm.

### Statistics

The results are expressed as the mean±SD. Statistical differences between two groups were determined by use of the two-tailed Student's t-test. In the case of more than 2 groups, differences were determined by ANOVA with Fisher's protected least significant difference.

### Results

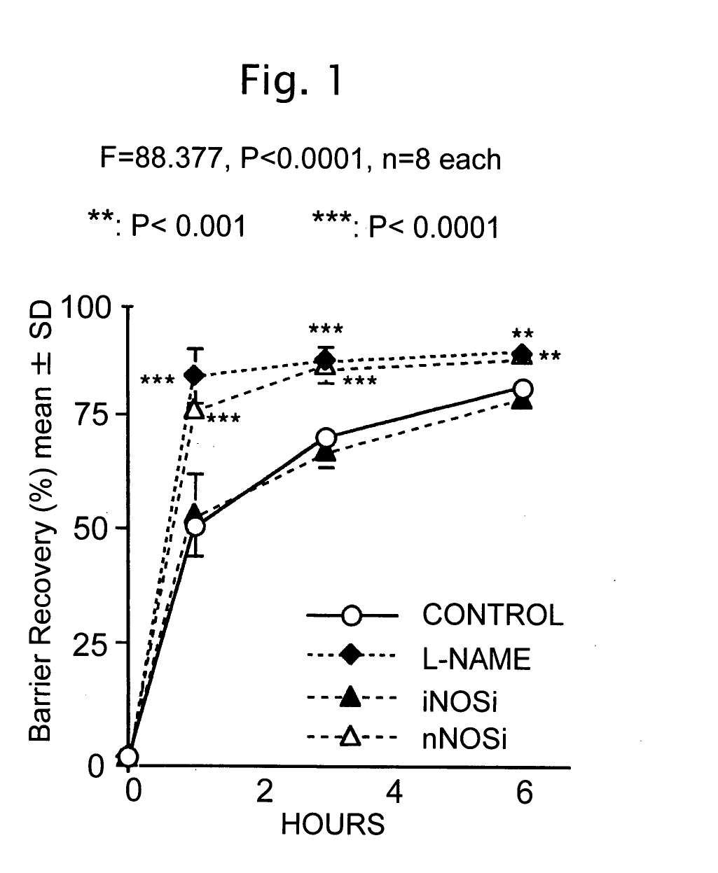

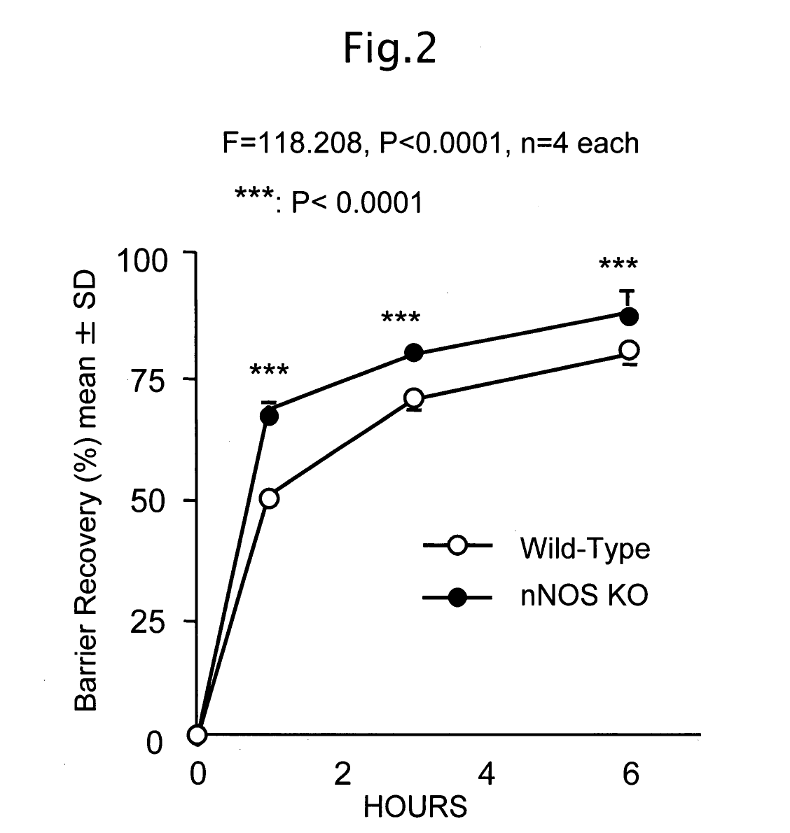

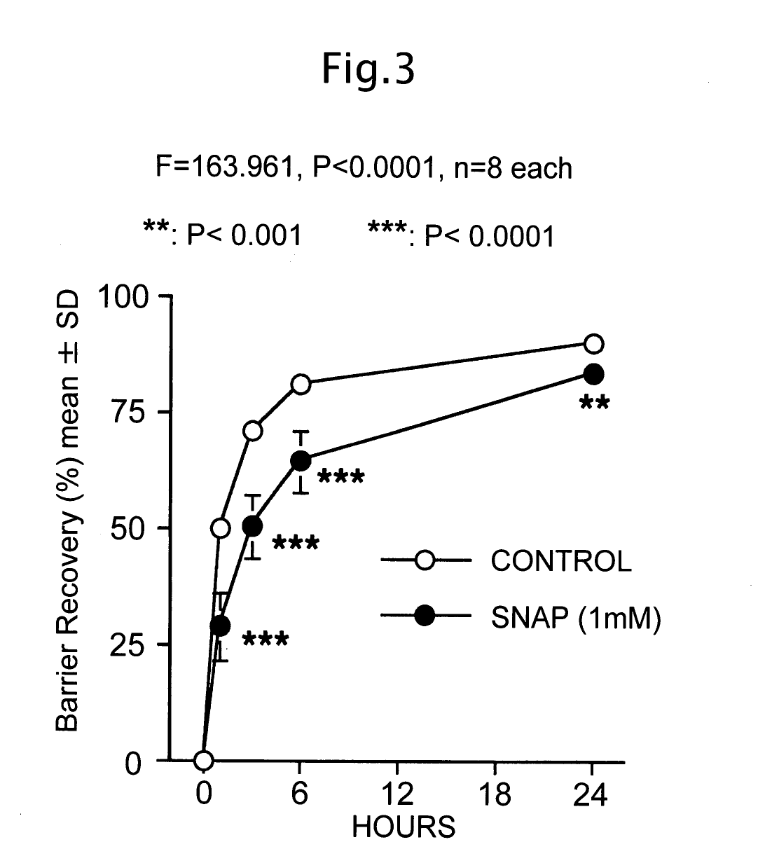

FIG. 1 shows the effects of topical application of NO synthase inhibitors on skin barrier recovery after tape stripping. Topical application of L-NAMA generally inhibits NO synthesis, and nNOS inhibitor (N^{ω}-propyl-L-arginine) accelerated the barrier repair. On the other hand, an iNOS inhibitor did not affect the barrier recovery. These effects were seen at 1, 3 and 6 hours after the treatment. Furthermore, to confirm the pharmacologic studies and definitively demonstrate the role of nNOS, we evaluated the barrier recovery in nNOS^{-/-} mice. Before barrier disruption, there was no significant difference in baseline between wild-type (21.25±1.32 mg/cm²) and nNOS^{-/-} mice (21.5±0.71 mg/cm²). After tape stripping, the barrier recovery in nNOS^{-/-} mice was significantly faster than in wild-type mice. (FIG. 2). FIG. 3 shows that topical application of the NO donor SNAP delayed skin barrier recovery. These results suggested that NO released from nNOS is involved in the processes of skin barrier recovery, and inhibition of nNOS activity specifically accelerates the recovery.

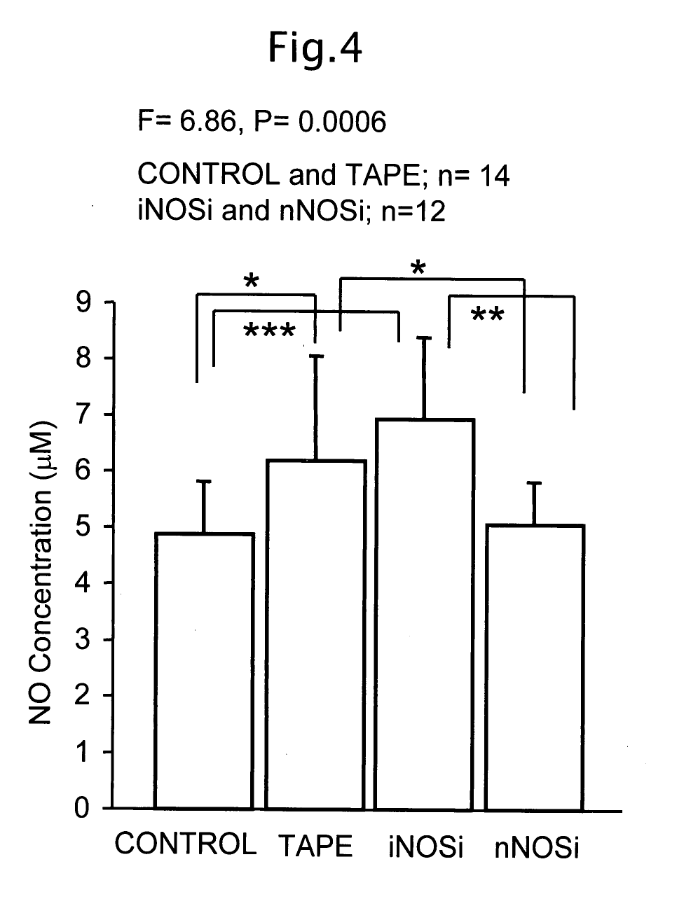

To examine the NO production or release upon barrier disruption, we examined the NO level in skin after tape stripping. FIG. 4 shows the NO production or release from skin organ culture with or without tape stripping. The NO level was increased at 30 min after the tape stripping. These results suggested that the NO level was increased immediately after barrier disruption. The increase was blocked by the presence of nNOS inhibitor. On the other hand, iNOS inhibitor did not affect (FIG. 4).

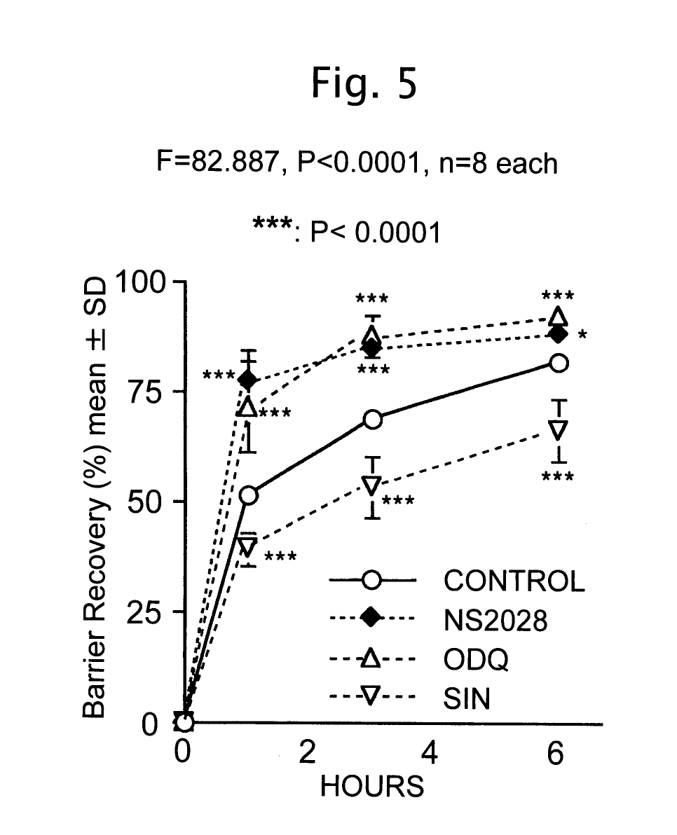

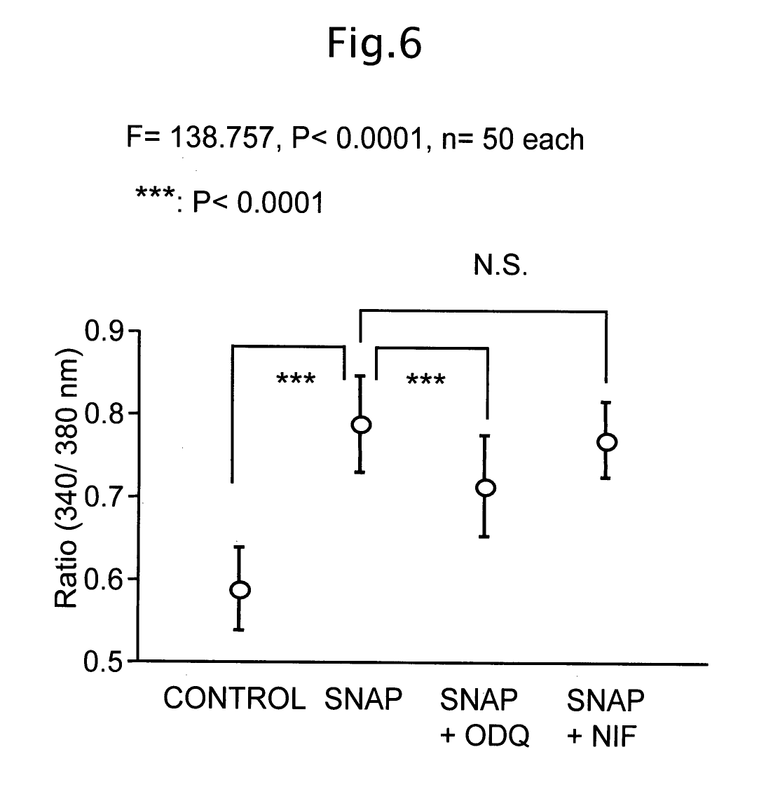

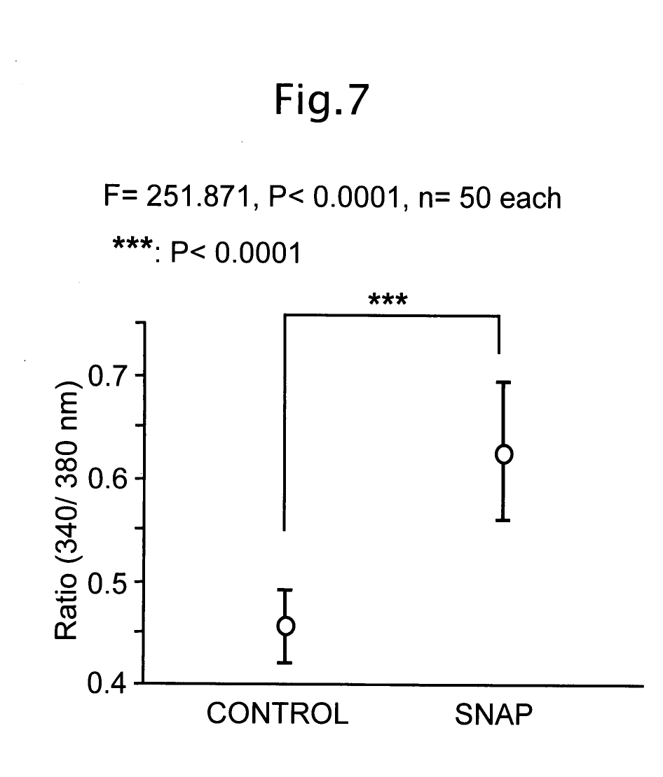

To ascertain the effect of the NO-cGMP pathway on barrier homeostasis, we examined the effects of guanylyl cyclase inhibitors and an activator on barrier recovery after tape stripping (FIG. 5). Topical application of NS 2028, which is a general inhibitor of guanylyl cyclase, and ODQ, which is a NO-sensitive guanylyl cyclase inhibitor, accelerated the barrier recovery. On the other hand, application of SIN, a guanylyl cyclase activator, delayed the barrier recovery. Intercellular calcium concentration, evaluated with fura-2AM, was increased in human cultured keratinocytes in the presence of 300 μM SNAP (FIG. 6). The increase induced by SNAP was blocked by 10 μM ODQ, while 50 μM nifedipine did not affect it (FIG. 6). In calcium-free medium, an increase of [Ca²⁺] was induced by SNAP (FIG. 7). These results suggest that the increase of Ca²⁺ induced by this NO donor might be a result of release of calcium from internal stores in endoplasmic reticulum.

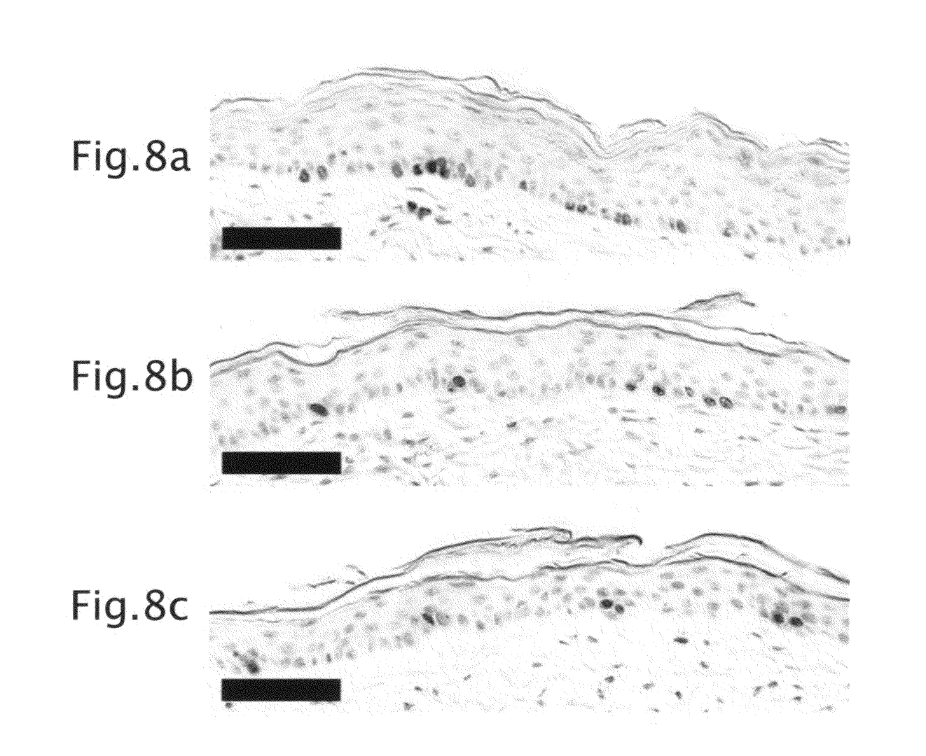

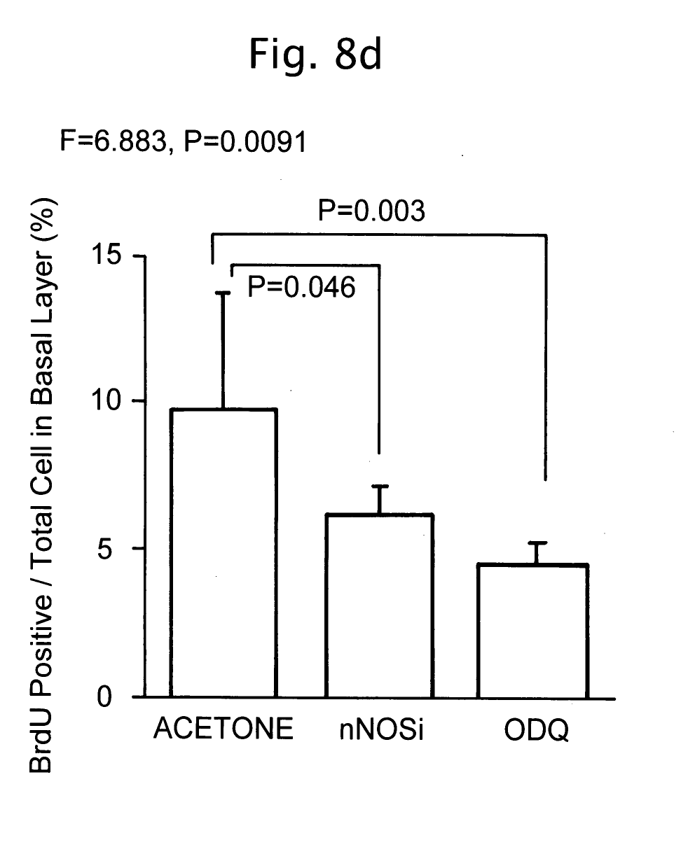

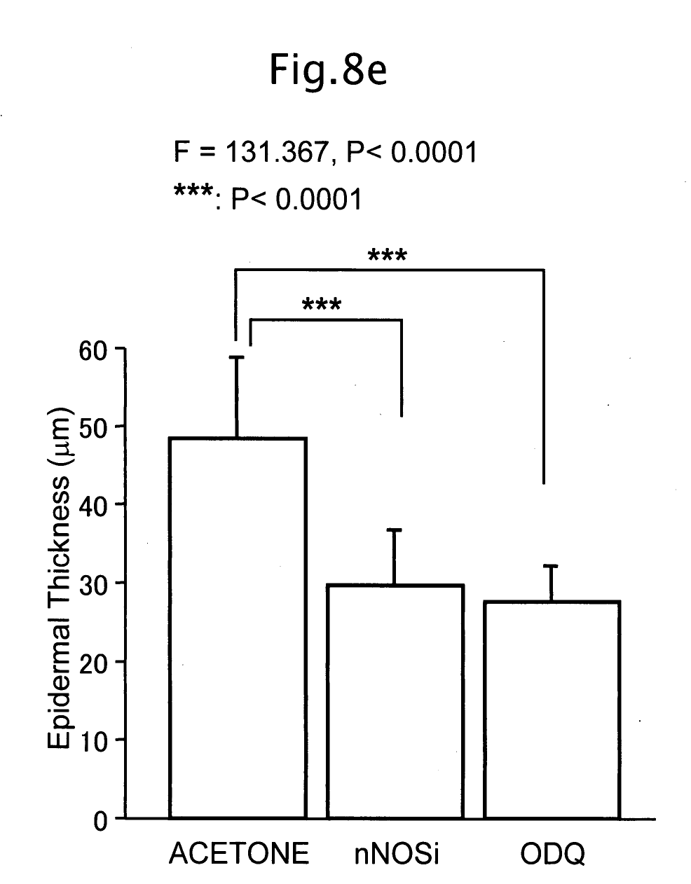

Topical application of a nNOS inhibitor and ODQ prevented the epidermal hyperplasia induced by acetone treatment under low environmental humidity. Representative sections are shown in FIG. 8. FIG. 8a shows the hyperproliferative epidermis treated with water after acetone treatment under low humidity. Dark spots represent BrdU-positive cells, which were increased on the epidermal basal layer (FIG. 8a). Topical application of 1 mM nNOS inhibitor prevented the epidermal hyperplasia (FIG. 8b). Application of 1 mM ODQ also prevented the hyperplasia (FIG. 8c). Quantified results are shown in FIGS. 8d and 8e. Significant reductions of epidermal proliferation and epidermal thickness were observed on both nNOS inhibitor-treated and ODQ-treated skin as compared with acetone treated skin.

### Discussion

In this study, we found that a nNOS inhibitor accelerated the barrier recovery rate, whereas an iNOS inhibitor had no effect on the barrier repair process. After tape stripping, the barrier recovery in nNOS^{-/-} mice was significantly faster than in wild-type mice. Topical application of a NO donor delayed barrier recovery in hairless mice. Thus, NO released from nNOS appears to play an important role in barrier recovery immediately after barrier disruption.

NO is produced by three isoforms of NOS. The constitutive isoforms, nNOS and eNOS, and the inducible isoform, iNOS, are all expressed in the skin(Bruch-Gerharz et al, 1998). Ormerod et al. observed strong nNOS staining in the granular layer of epidermis in normal skin, whereas eNOS staining was seen in the endothelium and weakly in the epidermis(Ormerod et al, 1998). They also demonstrated that nNOS was expressed throughout all levels of the epidermis in psoriatic lesions, and iNOS was significantly upregulated in psoriatic lesional skin, focally in keratinocytes. These findings suggest that nNOS and iNOS may play key roles in skin barrier homeostasis(Cals-Grierson and Ormerod, 2004). Thus, in the present study, we focused on nNOS and iNOS. But potentially, eNOS might also be involved in skin homeostasis. Further studies are needed to investigate the expression of each NOS isoform in the epidermis in relation to skin pathology.

In the present study, we evaluated NO release after tape stripping in an organ culture (FIG. 4). The tape-stripped skin showed a significant increase of NO release compared with untreated skin. The increase was blocked by nNOS inhibitor. However, iNOS inhibitor did not prevent the increase of NO release after tape stripping. nNOS is a calcium-dependent enzyme and increased levels of intercellular Ca²⁺ activate the enzyme via calmodulin, while iNOS is calcium-independent(Bruch-Gerharz et al, 1998). Changes in calcium occur in response to altered barrier function(Lee et al, 1992; Elias, 2005). Our results suggest that nNOS is activated after barrier disruption, and thus NO generated from nNOS rather than iNOS might be associated with skin barrier recovery. In normal human skin, nNOS was expressed in keratinocytes in the granular layer and eccrine sweat glands(Ormerod et al, 1998; Cals-Grierson and Ormerod, 2004; Sowden et al, 2005). Some studies have indicated that nNOS is involved in epidermal homeostasis. For example, NO derived from nNOS has been shown to be involved in the modulation of skin proliferation and melanogenesis(Romero-Graillet et al, 1997). It has also been demonstrated that nNOS is expressed in keratinocytes at a wound site(Boissel et al, 2004). Our data suggest an involvement of NO generated from nNOS in barrier repair.

NO activates guanylyl cyclase, resulting in cGMP production(Bredt and Snyder, 1989). The effects of NO in cellular signaling are related to its ability to regulate Ca²⁺ homeostasis through the activation of the NO-cGMP pathway(Clementi, 1998). In our studies, acceleration of the barrier recovery was induced by a NO-sensitive guanylyl cyclase inhibitor, whereas the enhancement of cGMP synthesis delayed barrier recovery (FIG. 5). These results suggest that the NO-cGMP pathway could be involved in skin barrier homeostasis. We previously demonstrated that influx of calcium into epidermal keratinocytes blocked lamellar body secretion and delayed barrier recovery(Denda et al, 2003). Previous studies suggested that cGMP affects the voltage-gated calcium channel in aortic smooth muscle cells (Kaur et al, 1998) and ganglion cells(Hirooka et al, 2000). Jiang et al. demonstrated that the store-operated Ca²⁺ entry pathway is regulated by cGMP in human hepatoma cells(Jiang et al, 2001). In the present study, treatment of primary human keratinocyte cultures with the NO donor SNAP increased the ([Ca²⁺]i) (FIG. 6). The increase was blocked by the guanylyl cyclase inhibitor ODQ (FIG. 6). However, the calcium channel blocker nifedipine did not prevent the increase of [Ca²⁺]i induced by the NO donor (FIG. 6). FIG. 7 shows the change in [Ca²⁺]i of primary human keratinocyte cultured in calcium-free medium. Treatment of the keratinocyte cultures with the NO donor SNAP increased the [Ca²⁺]i (FIG. 7). These results suggest that the increase of Ca²⁺ induced by this NO donor might be attributed not to the influx of Ca²⁺ through voltage-gated calcium channels, but to the release of calcium from the internal store in endoplasmic reticulum. Some signaling pathways that induce the release of calcium from the internal store in keratinocytes have been described(Rosenbach et al, 1993; Aoyama et al, 1995; Biro et al, 1998). Further studies are needed to investigate the signal pathway of intracellular calcium release in the present case.

Topical application of the nNOS inhibitor N^{ω}-propyl-L-arginine or ODQ reduced the epithelial hyperproliferative response induced by acetone treatment under low environmental humidity (FIG. 8). Treatments that accelerate barrier repair tend to prevent epidermal hyperplasia(Denda et al, 1997; Ashida et al, 2001). Barrier disruption induced epidermal DNA synthesis(Proksch et al, 1991). However, the calcium influx is also related to keratinocyte proliferation, differentiation, and inflammatory responses(Yuspa et al, 1988). During the terminal differentiation of keratinocytes, lipid synthesis and cornified envelope formation are also induced by calcium(Watanabe et al, 1998). Rossi et al. have shown that NO inhibits cornified envelope formation in human keratinocytes(Rossi et al, 2000). NO regulates the synthesis of gene products involved in keratinocyte differentiation and ceramide metabolism(Gallala et al, 2004). NO might play important roles in the regulation of various physiological events in skin homeostasis, via regulation of calcium concentration in keratinocytes.

We speculate that barrier disruption activated nNOS in the present experiments, and increased the NO level in the epidermis. The NO increase induced cGMP generation, leading to the release of calcium from the internal store in epidermal keratinocytes. Consequently, the intracellular calcium level increased in the epidermal keratinocytes and delayed barrier repair. If this cascade is blocked, e.g., by inhibition of NO synthesis or guanylyl cyclase, barrier recovery is accelerated. If these ideas are correct, there are implications for new clinical methodology to treat dermatoses such as psoriasis and atopic disease, which are characterized by barrier dysfunction and epidermal hyperplasia.

### REFERENCES

Aoyama, Y., M. Seishima, S. Mori, Y. Kitajima, Y. Okano and Y. Nozawa: Involvement of protein kinase C in bradykinin-induced intracellular calcium increase in primary cultured human keratinocytes. J Dermatol Sci 9(2): 111-6, 1995
Ashida, Y., M. Denda and T. Hirao: Histamine H1 and H2 receptor antagonists accelerate skin barrier repair and prevent epidermal hyperplasia induced by barrier disruption in a dry environment. J Invest Dermatol 116(2): 261-5, 2001
Biro, T., I. Szabo, L. Kovacs, J. Hunyadi and L. Csernoch: Distinct subpopulations in HaCaT cells as revealed by the characteristics of intracellular calcium release induced by phosphoinositide-coupled agonists. Arch Dermatol Res 290(5): 270-6, 1998
Boissel, J. P., D. Ohly, M. Bros, U. Godtel-Armbrust, U. Forstermann and S. Frank: The neuronal nitric oxide synthase is upregulated in mouse skin repair and in response to epidermal growth factor in human HaCaT keratinocytes. J Invest Dermatol 123(1): 132-9, 2004
Bredt, D. S. and S. H. Snyder: Nitric oxide mediates glutamate-linked enhancement of cGMP levels in the cerebellum. Proc Natl Acad Sci USA 86(22): 9030-3, 1989
Bruch-Gerharz, D., T. Ruzicka and V. Kolb-Bachofen: Nitric oxide in human skin: current status and future prospects. J Invest Dermatol 110(1): 1-7, 1998
Cals-Grierson, M. M. and A. D. Ormerod: Nitric oxide function in the skin. Nitric Oxide 10(4): 179-93, 2004
Clementi, E.: Role of nitric oxide and its intracellular signalling pathways in the control of Ca2+ homeostasis. Biochem Pharmacol 55(6): 713-8, 1998
Denda, M., S. Fuziwara and K. Inoue: Influx of calcium and chloride ions into epidermal keratinocytes regulates exocytosis of epidermal lamellar bodies and skin permeability barrier homeostasis. J Invest Dermatol 121(2): 362-7, 2003
Denda, M., K. Kitamura, P. M. Elias and K. R. Feingold: trans-4-(Aminomethyl)cyclohexane carboxylic acid (T-AMCHA), an anti-fibrinolytic agent, accelerates barrier recovery and prevents the epidermal hyperplasia induced by epidermal injury in hairless mice and humans. J Invest Dermatol 109(1): 84-90, 1997
Denda, M., J. Sato, T. Tsuchiya, P. M. Elias and K. R. Feingold: Low humidity stimulates epidermal DNA synthesis and amplifies the hyperproliferative response to barrier disruption: implication for seasonal exacerbations of inflammatory dermatoses. J Invest Dermatol 111(5): 873-8, 1998
Denda, M. and T. Tsuchiya: Barrier recovery rate varies time-dependently in human skin. Br J Dermatol 142(5): 881-4, 2000
Elias, P. M.: Stratum corneum defensive functions: an integrated view. J Invest Dermatol 125(2): 183-200, 2005
Elias, P. M. and K. R. Feingold: Coordinate regulation of epidermal differentiation and barrier homeostasis. Skin Pharmacol Appl Skin Physiol 14 Suppl 1: 28-34, 2001
Gallala, H., O. Macheleidt, T. Doering, V. Schreiner and K. Sandhoff: Nitric oxide regulates synthesis of gene products involved in keratinocyte differentiation and ceramide metabolism. Eur J Cell Biol 83(11-12): 667-79, 2004
Giustizieri, M. L., C. Albanesi, C. Scarponi, O. De Pita and G. Girolomoni: Nitric oxide donors suppress chemokine production by keratinocytes in vitro and in vivo. Am J Pathol 161(4): 1409-18, 2002
Grynkiewicz, G., M. Poenie and R. Y. Tsien: A new generation of Ca2+ indicators with greatly improved fluorescence properties. J Biol Chem 260(6): 3440-50, 1985
Hirooka, K., D. E. Kourennyi and S. Barnes: Calcium channel activation facilitated by nitric oxide in retinal ganglion cells. J Neurophysiol 83(1): 198-206, 2000
Horn, T. F., G. Wolf, S. Duffy, S. Weiss, G. Keilhoff and B. A. MacVicar: Nitric oxide promotes intracellular calcium release from mitochondria in striatal neurons. Faseb J 16(12): 1611-22, 2002
Jiang, J. L., M. K. Yu, Z. N. Chen and H. C. Chan: cGMP-regulated store-operated calcium entry in human hepatoma cells. Cell Biol Int 25(10): 993-5, 2001
Kaur, K., J. Yao, X. Pan, C. Matthews and A. Hassid: NO decreases phosphorylation of focal adhesion proteins via reduction of Ca in rat aortic smooth muscle cells. Am J Physiol 274(5 Pt 2): H1613-9, 1998
Koizumi, S., H. Uneyama, M. Ikeda, S. Ueno and K. Inoue: Inhibition by imipramine of ATP-evoked responses in rat pheochromocytoma cells. Biochem Biophys Res Commun 244(2): 342-6, 1998
Lau, K. S., R. W. Grange, E. Isotani, I. H. Sarelius, K. E. Kamm, P. L. Huang and J. T. Stull: nNOS and eNOS modulate cGMP formation and vascular response in contracting fast-twitch skeletal muscle. Physiol Genomics 2(1): 21-7, 2000
Lee, S. H., P. M. Elias, E. Proksch, G. K. Menon, M. Mao-Quiang and K. R. Feingold: Calcium and potassium are important regulators of barrier homeostasis in murine epidermis. J Clin Invest 89(2): 530-8, 1992
Ormerod, A. D., R. Weller, P. Copeland, N. Benjamin, S. H. Ralston, P. Grabowksi and R. Herriot: Detection of nitric oxide and nitric oxide synthases in psoriasis. Arch Dermatol Res 290(1-2): 3-8, 1998
Proksch, E., K. R. Feingold, M. Q. Man and P. M. Elias: Barrier function regulates epidermal DNA synthesis. J Clin Invest 87(5): 1668-73, 1991
Romero-Graillet, C., E. Aberdam, M. Clement, J. P. Ortonne and R. Ballotti: Nitric oxide produced by ultraviolet-irradiated keratinocytes stimulates melanogenesis. J Clin Invest 99(4): 635-42, 1997
Rosenbach, T., C. Liesegang, S. Binting and B. M. Czarnetzki: Inositol phosphate formation and release of intracellular free calcium by bradykinin in HaCaT keratinocytes. Arch Dermatol Res 285(7): 393-6, 1993
Rossi, A., M. V. Catani, E. Candi, F. Bernassola, P. Puddu and G. Melino: Nitric oxide inhibits cornified envelope formation in human keratinocytes by inactivating transglutaminases and activating protein 1. J Invest Dermatol 115(4): 731-9, 2000
Sowden, H. M., K. M. Naseem and D. J. Tobin: Differential expression of nitric oxide synthases in human scalp epidermal and hair follicle pigmentary units: implications for regulation of melanogenesis. Br J Dermatol 153(2): 301-9, 2005
Taniuchi, S., T. Kojima, K. Hara Mt, A. Yamamoto, M. Sasai, H. Takahashi and Y. Kobayashi: Increased serum nitrate levels in infants with atopic dermatitis. Allergy 56(7): 693-5, 2001
Watanabe, R., K. Wu, P. Paul, D. L. Marks, T. Kobayashi, M. R. Pittelkow and R. E. Pagano: Up-regulation of glucosylceramide synthase expression and activity during human keratinocyte differentiation. J Biol Chem 273(16): 9651-5, 1998
Weller, R.: Nitric oxide: a key mediator in cutaneous physiology. Clin Exp Dermatol 28(5): 511-4, 2003
Yuspa, S. H., H. Hennings, R. W. Tucker, S. Jaken, A. E. Kilkenny and D. R. Roop: Signal transduction for proliferation and differentiation in keratinocytes. Ann N Y Acad Sci 548: 191-6, 1988

What is claimed is:

1. A method for accelerating cutaneous barrier recovery by inhibiting production of Nitric Oxide by an epidermal cell.

2. The method of claim 1, wherein inhibition of the production of Nitric Oxide by an epidermal cell is attained by applying Nitric Oxide Synthase (NOS), neural Nitric Oxide Synthase (nNOS) inhibitor, Nitric Oxide sensitive guanylyl cyclase inhibitor or general cyclase inhibitor on skin.

3. A method for preventing epidermal hyperplasia induced by barrier disruption by inhibiting production of Nitric Oxide by an epidermal cell.

4. The method of claim 3, wherein inhibition of the production of Nitric Oxide by an epidermal cell is attained by applying Nitric Oxide Synthase (NOS), neural Nitric Oxide Synthase (nNOS) inhibitor, Nitric Oxide sensitive guanylyl cyclase inhibitor or general cyclase inhibitor on skin.
